(19) United States
(12) Patent Application Publication
(10) Pub. No.: US 2023/0310883 A1
(43) Pub. Date: Oct. 5, 2023

(54) WEARABLE SAFETY LASING LIGHT THERAPY DEVICE

(71) Applicant: Taiwan RedEye Biomedical Inc., Hsinchu City (TW)

(72) Inventors: Tsung-Jui LIN, Hsinchu City (TW); Chen-Chung CHANG, Hsinchu City (TW); Shuo-Ting YAN, Hsinchu City (TW)

(21) Appl. No.: 17/711,232

(22) Filed: Apr. 1, 2022

(51) Int. Cl. A61N 5/067

(52) U.S. Cl. CPC A61N 5/067; A61N 2005/0626

(57) ABSTRACT

A shell of the present invention includes a light exiting surface with an opening; a power button is mounted on the shell; inside the shell, a proximity sensing module is mounted facing the light exiting surface, and a processing module is electrically connected to the proximity sensing module, the power switch, and a light emission module; when the power switch is switched on, the processing module enters a hibernation mode; when the processing module in hibernation mode receives a trigger signal sent by the proximity sensing module for sensing a human body in close proximity, the processing module exits the hibernation mode and controls the light emission module to generate a light therapy beam; the present invention ensures the opening is closely contacting the human body before generating the light therapy beam, decreasing risks of the light therapy beam exiting the opening and hitting human eyes directly.

### BACKGROUND OF THE INVENTION

### 1. Field of the Invention

The present invention relates to a lasing light therapy device, in particular to a wearable safety lasing light therapy device.

### 2. Description of the Related Art

Knee pain is a common nuisance in modern times. Knee pain may occur as some engage in sports and accidentally wound their knees, and may also occur as some gain weight and develop arthritis, rheumatoid arthritis (RA), or fibromyalgia in adulthood.

Currently, one of the ways to alleviate knee pain is to apply a photobiomodulation therapy to a person's knee. The photobiomodulation therapy is a light therapy that utilizes low energy laser beams to stimulate muscles or tissues around a joint to repair damages and alleviate pain. Therefore when the photobiomodulation therapy is applied to the person's knee, the knee pain will be alleviated.

However, even though low energy laser beam of long wavelengths is harmless to human skin, light intensity of the low energy laser beam is still too strong for human eyes. In other words, since unit surface area of the lasing spot receives too much energy from the laser, the laser would appear to be too bright for human eyes. When the laser directly hits the human eyes, the human eyes may be damaged.

Despite this, the current lasing device only has a start button to switch on laser. If a lasing device strapped on the user for photobiomodulation therapy accidentally goes loose while lasing, a light path of the laser beam would be unpredictable, prompting the possibility of directly shooting laser into the person's eyes. Furthermore, since the spectrum of the lasing light might be outside of the visible spectrum, if a person accidentally looks at the laser, the person would not be able to notice and react immediately, and this may lead to further damage to the eyes.

The circumstance described above is especially dangerous for a person with knee pain, because when the person adjusts the laser for conducting light therapy to the knee, the person might accidentally touch the start button and switch on the laser. Without fully adjusted to a designated lasing position, the laser might accidentally and directly shine into the person's eyes looking at the knee.

### SUMMARY OF THE INVENTION

Regarding the aforementioned problems, the present invention provides a wearable safety lasing light therapy device. The wearable safety lasing light therapy device ensures lasing would only be initiated once the present invention is properly settled at a designated lasing position.

The wearable safety lasing light therapy device includes a shell, a power switch, a light emission module, a processing module, and a proximity sensing module.

The shell includes a light exiting surface, and the light exiting surface includes an opening.

The power switch is mounted on the shell.

The light emission module is mounted within the shell and faces the light exiting surface, and is adapted to produce a light therapy beam. The light therapy beam shoots out of the shell through the opening.

The processing module is mounted within the shell, and electrically connects the light emission module and the power switch.

The proximity sensing module is mounted within the shell and faces the light exiting surface, and electrically connects the processing module.

When the power switch is switched on, the power switch generates a power signal to the processing module.

When the proximity sensing module senses a human body is in close proximity to the light exiting surface, the proximity sensing module generates a trigger signal and sends the trigger signal to the processing module.

When the processing module receives the power signal, the processing module enters a hibernation mode. When the processing module is in the hibernation mode and receives the trigger signal, the processing module exits the hibernation mode, enters an emission mode, and controls the light emission module to produce the light therapy beam.

When the present invention is worn and trapped around a person's knee, the light emission module is able to produce the light therapy beam safely without shining the beam elsewhere than the knee. The proximity sensing module of the present invention acts as a safety mechanism. When the proximity sensing module senses the human body is in close proximity to the light exiting surface, the proximity sensing module generates the trigger signal. It indicates that the opening is in a designated position of closely contacting the human body, for instance the person's knee. Then, the light therapy beam produced by the light emission module therefore is safely contained without risks of directly shooting at the person's eyes. In other words, the processing module needs to first confirm the light emission module is in close proximity to the person's knee through the proximity sensing module before generating the light therapy beam through the light emission module. This way the present invention is able to drastically decrease risks of the light therapy beam shooting directly into the person's eyes.

### BRIEF DESCRIPTION OF THE DRAWINGS

FIG. 1 is a perspective view of a shell of a wearable safety lasing light therapy device of the present invention.

FIG. 2 is another perspective view of the shell of the wearable safety lasing light therapy device of the present invention.

FIG. 3 is an exploded view of the wearable safety lasing light therapy device of the present invention.

FIG. 4 is a cross-sectional view of the wearable safety lasing light therapy device of the present invention.

FIG. 5 is a block diagram of the wearable safety lasing light therapy device of the present invention.

FIG. 6A is another cross-sectional view of the safe lasing wearable light therapy device of the present invention.

FIG. 6B is an enlarged cross-sectional view of the safe lasing wearable light therapy device of the present invention.

FIG. 6C is another enlarged cross-sectional view of the safe lasing wearable light therapy device of the present invention.

FIG. 7 is a perspective view of an application of the wearable safety lasing light therapy device of the present invention.

FIG. 8 is a perspective view of another application of the wearable safety lasing light therapy device of the present invention.

FIG. 9 is a perspective view of how the wearable safety lasing light therapy device of the present invention is applied to a person's knee.

### DETAILED DESCRIPTION OF THE INVENTION

The present invention provides a wearable safety lasing light therapy device. In a first embodiment of the present invention, the wearable safety lasing light therapy device includes a shell, a control circuit board, a light emission circuit board, and a battery.

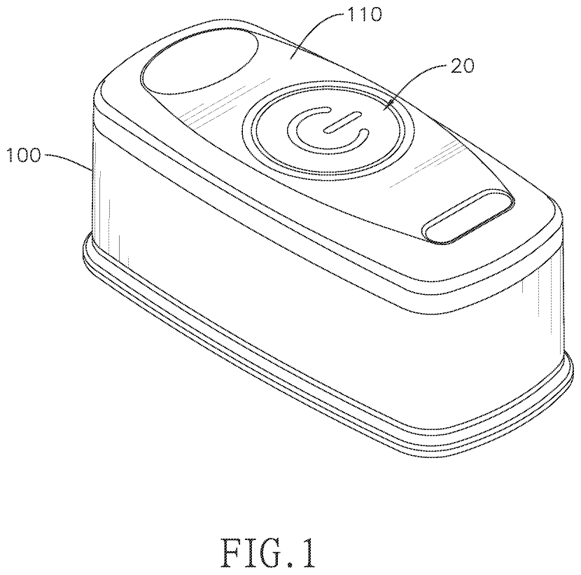

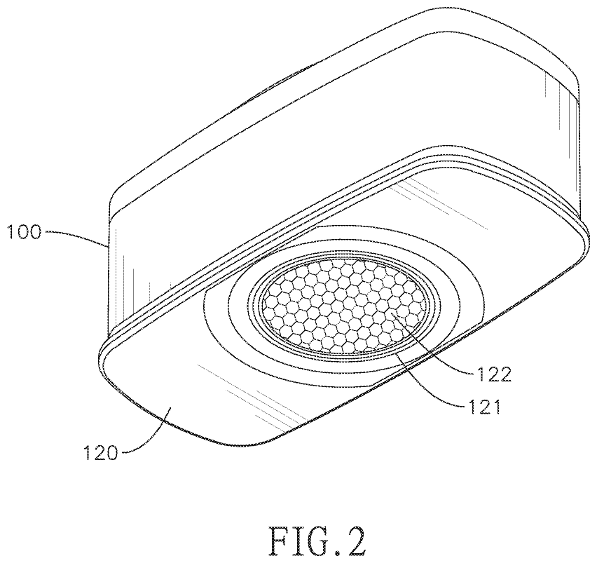

With reference to FIG. 1, a power switch 20 is mounted on the shell 100. The shell 100 includes a front surface 110 and a light exiting surface 120. The light exiting surface 120 includes an opening 121. A transparent cover 122 is mounted on the light exiting surface 120 of the shell 100, and more particularly inside the opening 121.

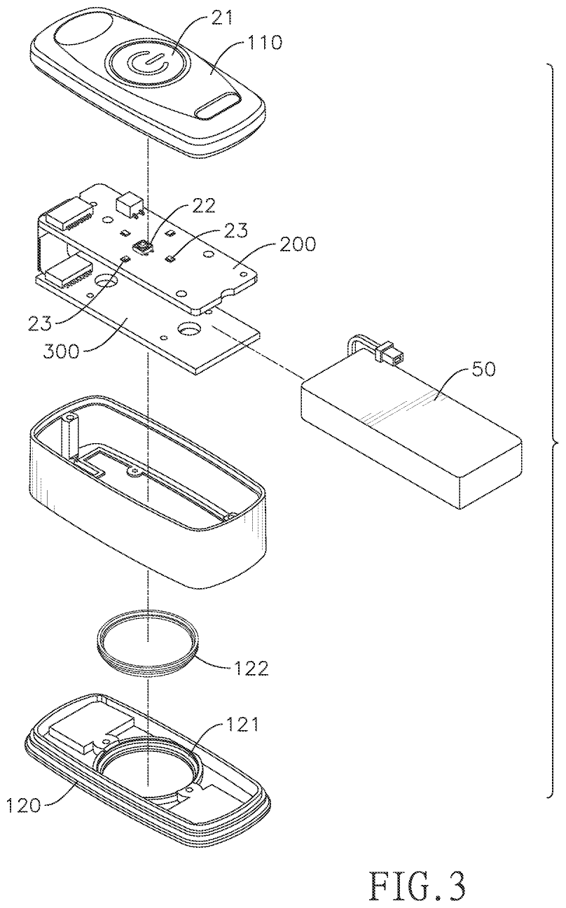

With reference to FIGS. 3 to 5, the control circuit board 200, the light emission circuit board 300, and the battery 50 are mounted respectively within the shell 100, in other words between the front surface 110 and the light exiting surface 120.

Furthermore, the power switch 20 includes a push button cover 21 and a push button switch 22. The push button cover 21 is mounted on the front surface 110 of the shell 100. The push button switch 22 and a processing module 10 are respectively mounted on the control circuit board 200, and the push button switch 22 is electrically connected to the processing module 10. A light emission module 30 and a proximity sensing module 40 are respectively mounted on the light emission circuit board 300, and are also respectively facing the light exiting surface 120 of the shell 100. The battery 50 is mounted between the control circuit board 200 and the light emission circuit board 300. The battery 50 is electrically connected to the processing module 10 and the light emission module for providing electricity.

The processing module 10 is electrically connected to the power switch 20, the light emission module 30, and the proximity sensing module 40. When the power switch 20 is switched on, in other words when the push button cover 21 and the push button switch 22 are pressed, the push button switch 22 of the power switch 20 generates a power signal and delivers the power signal to the processing module 10. When the processing module 10 receives the power signal, the processing module 10 enters a hibernation mode.

When the proximity sensing module 40 senses a human body is in close proximity to the light exiting surface 120, the proximity sensing module 40 generates a trigger signal and sends the trigger signal to the processing module 10. When the processing module 10 is in the hibernation mode and receives the trigger signal, the processing module 10 exits the hibernation mode, enters an emission mode, and controls the light emission module 30 to produce a light therapy beam.

The light therapy beam produced by the light emission module 30 exits the light exiting surface 120 through the opening 121, passing through the transparent cover 122. Since the light emission circuit board 300 closely contacts the light exiting surface 120, the light therapy beam travels only little distance to the opening 121 before exiting the light exiting surface 120. This way optical loss of the light therapy beam can be minimized. The transparent cover 122 mounted between the light emission module 30 on the light emission circuit board 300 and the opening 121 of the light exiting surface 120 is able to keep dust and external pollutant away from the light emission module 30.

In the present embodiment, the light exiting surface 120 of the shell 100 is an insulator. The proximity sensing module 40 is a capacitive proximity sensor with a sensor electrode. More particularly, the light exiting surface 120 is a dielectric, or an electrical insulator that can be polarized by an applied electric field. Since the proximity sensing module 40 closely contacts the light exiting surface 120 along with the light emission circuit board 300, the sensor electrode also closely contacts the light exiting surface 120. When the human body contacts a part of the light exiting surface 120 of the shell 100 corresponding to the sensor electrode, the capacitive proximity sensor generates the trigger signal and sends the trigger signal to the processing module 10.

In another embodiment, the proximity sensing module 40 is a resistive sensor, and the processing module 10 also stores a first threshold. When the light exiting surface 120 is touched and deformed by a user, a distance between the light exiting surface 120 and the resistive sensor becomes smaller because of the deformation. When the distance between the light exiting surface 120 and the resistive sensor becomes smaller than the first threshold, the resistive sensor generates the trigger signal and sends the trigger signal to the processing module 10.

In yet another embodiment, the proximity sensing module 40 is an optical sensor. The optical sensor generates a sensing signal and shoots the sensing signal out of the transparent cover 122 for sensing the human body. When the human body approaches the light exiting surface 120, the optical sensor generates a distance signal and sends the distance signal to the processing module 10. The processing module 10 also stores a distance threshold. When the processing module 10 determines the distance signal is less than or equal to the distance threshold, the processing module 10 only then determines to receive the trigger signal from the proximity sensing module 40. When the processing module 10 determines the distance signal is greater than the distance threshold, the processing module 10 determines yet to receive the trigger signal from the proximity sensing module 40.

Further, the power switch 20 also includes a backlight light source 23. The backlight light source 23 is mounted on the control circuit board 200, and the backlight light source 23 faces the front surface 110 of the shell 100 with the push button cover 21. When the processing module 10 enters the hibernation mode, the processing module 10 controls the backlight light source 23 of the power switch 20 to generate a first light signal. When the processing module 10 enters the emission mode, the processing module 10 controls the backlight light source 23 of the power switch 20 to generate a second light signal. The first light signal and the second light signal have different colors or different blinking frequencies.

In FIG. 3, the backlight light source 23 further includes four light-emitting diodes (LEDs). When the processing module 10 enters the hibernation mode, the processing module 10 controls the backlight light source 23 to generate green light. When the processing module 10 enters the emission mode, the processing module 10 controls the backlight light source 23 to generate blue light. In other words, the first light signal is green light, and the second light signal is blue light.

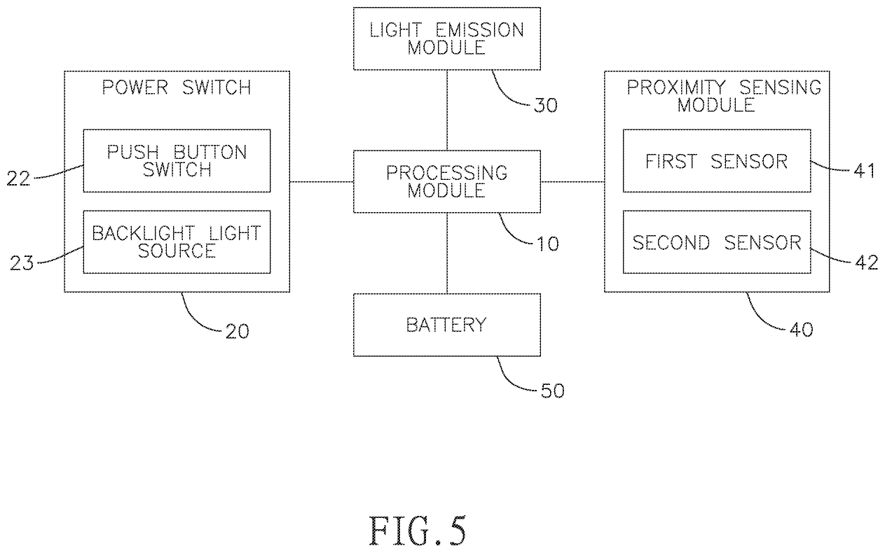

With further reference to FIG. 5, the proximity sensing module 40 on the light emission circuit board 300 further includes a first sensor 41 and a second sensor 42. The first sensor 41 and the second sensor 42 are respectively mounted on two opposite sides of the light emission circuit board 300. The first sensor 41 and the second sensor 42 also face the light exiting surface 120. When the first sensor 41 senses the human body is in close proximity to the light exiting surface 120, the first sensor 41 generates a first trigger signal and sends the first trigger signal to the processing module 10. When the second sensor 42 senses the human body is in close proximity to the light exiting surface 120, the second sensor 42 generates a second trigger signal and sends the second trigger signal to the processing module 10. When the processing module 10 is in the hibernation mode and the processing module 10 simultaneously receives the first trigger signal and the second trigger signal, the processing module 10 only then determines to receive the trigger signal from the proximity sensing module 40. However, when the processing module 10 is in the hibernation mode and the processing module 10 is yet to receive the first trigger signal and the second trigger signal simultaneously, for example only receiving the first trigger signal without receiving the second trigger signal, the processing module 10 stays in the hibernation mode.

After the processing module 10 enters the emission mode, the processing module 10 further determines whether receiving the first trigger signal or the second trigger signal. When the processing module 10 determines receiving the first trigger signal or the second trigger signal, the processing module 10 stays in the emission mode. When the processing module 10 determines missing the first trigger signal and the second trigger signal, the processing module 10 exits the emission mode and enters back to the hibernation mode. This way the processing module 10 would control the LEDs of the backlight light source 23 to change from generating blue light to generating green light.

After the processing module 10 enters the emission mode and determines receiving the first trigger signal or the second trigger signal, for example just receiving the first trigger signal, the processing module 10 determines that the opening 121 is only slightly shifted. Since the opening 121 is only slightly shifted, the light therapy beam is still free from shining directly at eyes of the user, and thus the processing module 10 continues to control the light emission module 30 to generate the light therapy beam.

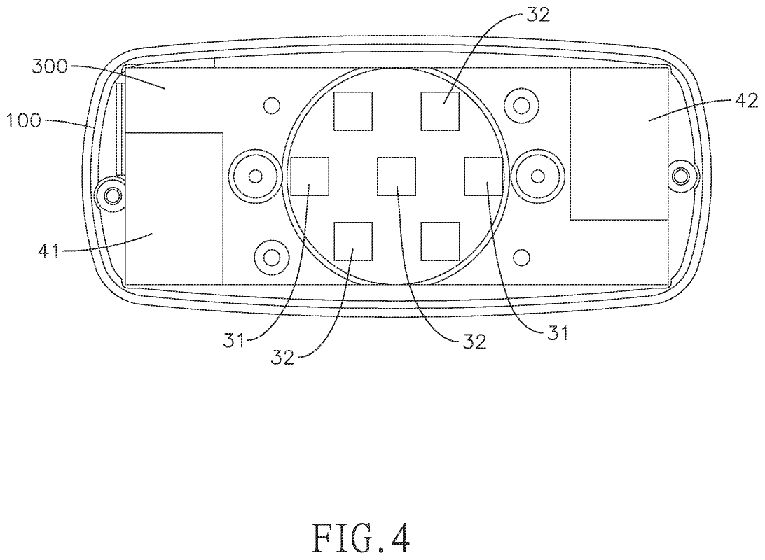

The light emission module 30 further includes at least one visible light unit 31 and at least one non-visible light unit 32. In FIG. 4, the light emission module 30 includes two visible light units 31 and five non-visible light units 32. When the processing unit 30 controls the light emission module 30 to generate the light therapy beam, the at least one non-visible light unit 32 generates a laser beam, and the at least one visible light unit 31 generates a colored beam. The colored beam consists of light in visual spectrum, and the laser beam consists of light outside of visual spectrum. Furthermore, the colored light is a diverging light. The diverging light causes a shined surface to have a big light spot. The visible light units 31 are LEDs, and the non-visible light units 32 are laser diodes (LDs).

In the emission mode, the processing module 10 controls the light emission module 30 to generate continuous waves or pulses of the light therapy beam. The pulsed light therapy beam has a duty cycle of 50%. Also, the processing module 10 includes an additional timer and stores a working time threshold.

When the processing module 10 enters the emission mode, the processing module 10 starts counting a working time. The processing module 10 further determines whether the working time is greater than or equal to the working time threshold. When the processing module 10 determines that the working time is greater than or equal to the working time threshold, the processing module 10 automatically stops generating the laser beam and switches off. When the processing module 10 determines that the working time is less than the working time threshold, the processing module 10 continues generating the laser beam. After the processing module 10 switches off, the processing module 10 needs to once again receive the power signal to switch on and enter the hibernation mode. In the present embodiment, the working time threshold is 10 minutes.

The working time threshold actually corresponds to how long a timed light therapy program lasts. The timed light therapy program, for instance, lasts for 10 minutes. In a hypothetical situation, when a user accidentally interrupts the timed light therapy program under the emission mode, the user might hold different expectations toward the present invention. For instance, suppose the present invention is closely contacting a knee of the user for conducting light therapy to the knee. The user however moves the knee and causes the proximity sensing module 40 of the present invention to briefly move away from the knee and briefly stop producing the light therapy beam. In this situation, the user might hold two different expectations after the interruption of the light therapy beam. One expectation is hoping the timed light therapy program will start from the beginning in order to fully execute the timed light therapy program. The other expectation is hoping the timed light therapy program will continue from the interruption, and proceed further to fully execute the timed light therapy program. In other words, the user might hold different understanding and expectations toward a full execution of the timed light therapy program. The present invention therefore provides two variations of embodiments to satisfy the user's expectations.

In one embodiment, the processing module 10 determines missing the first trigger signal and the second trigger signal, and thus the processing module 10 enters the hibernation mode and resets the working time. When the processing module 10 receives the trigger signal from the proximity sensing module 40 and returns to the emission mode, the processing module 10 starts counting the working time again from the beginning. This way the time light therapy program is being executed from the beginning again.

In another embodiment, the processing module 10 determines missing the first trigger signal and the second trigger signal, and thus the processing module 10 enters the hibernation mode and pauses counting the working time. When the processing module 10 receives the trigger signal from the proximity sensing module 40 and returns to the emission mode, the processing module 10 stops the pause of counting the working time, allowing the working time to count further. In other words, when the processing module 10 pauses counting the working time, a progress of the timed light therapy program is also paused, and when the processing module 10 stops the pause of counting the working time, the progress of the timed light therapy program is continued.

Furthermore, the non-visible light units 32 are mounted together at a center of the light emission circuit board 300, and the two visible light units 31 are mounted on opposite sides of the non-visible light units 32. This ensures light spots of the colored beam generated by the visible light units 31 cover light spots of the laser beam generated by the non-visible light units 32 across all angles. In other words, a huge light spot on a surface shined by the visible light units 31 encompasses smaller areas on the surface shined by the non-visible light units 32. For this reason, even if the user fails to register the laser beam, the user would still be able to see the huge light spot shined by the visible light units 31 and know where the laser beam is shining. This helps to prevent accidentally looking at the laser beam.

The human eyes can see lights with spectrum around 360 nanometers (nm) to 830 nm. In the present embodiment, the laser beam generated by the light emission module 30 has spectrum from 600 nm to 940 nm. In other words, the human eyes would fail to perceive light of 830 nm to 940 nm, or the infrared wavelength. This is why it is necessary for the present invention to implement the visible light units 31 to help notifying the user where the laser beam is shining, and to avoid having the user accidentally looking at the laser beam.

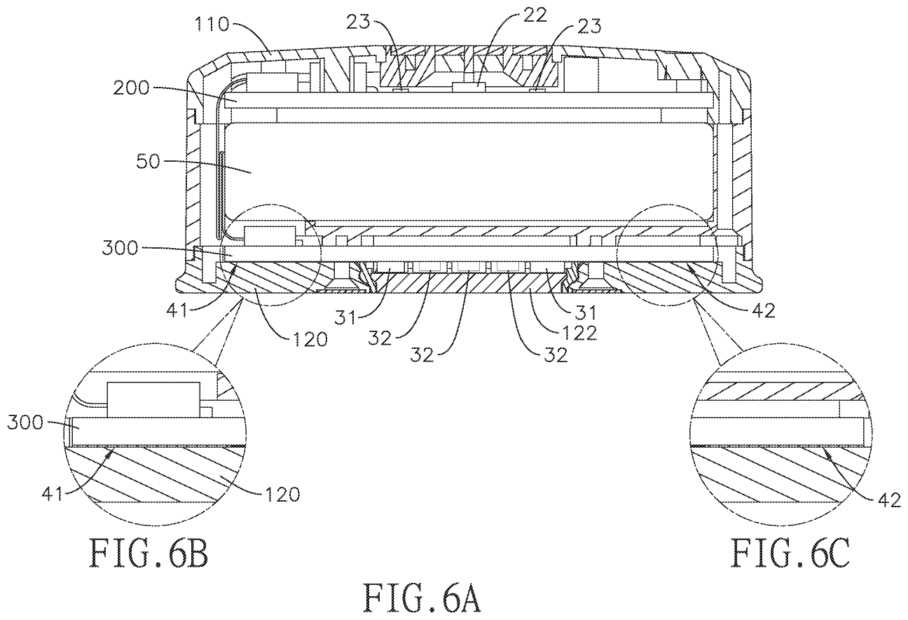

With further reference to FIGS. 6A to 6C, from a cross-sectional view of the present embodiment, the light emission circuit board 300 closely contacts the light exiting surface 120. As a result, when the light exiting surface 120 is in close proximity to the human body, the first sensor 41 and the second sensor 42 will be able to easily sense the human body and accordingly generate the first trigger signal and the second trigger signal.

In the present embodiment, a thickness of a wall of the light exiting surface 120 of the shell 100 is less than 3 millimeters (mm). This way, the light exiting surface 120 is thin enough to allow the first sensor 41 and the second sensor 42 of the proximity sensing module 40 to sense touches of the human body. The human body of the user is a conductor. The capacitive proximity sensor is used for sensing capacitive coupling between the sensor electrode and the human skin to determine whether the sensor electrode is touching or in close proximity to the human skin. To be more specific, the light exiting surface 120 is a dielectric located between two conductors—one conductor being the human body and the other conductor being the sensor electrode of the capacitive proximity sensor. This way a capacitor is created between the human body, the light exiting surface 120, and the sensor electrode. Furthermore, an electric field distribution of the capacitor would change depending on whether the human body touches or approaches the light exiting surface 120. When the human body touches or approaches the light exiting surface 120, a total of charges on the sensor electrode increases, so the electric field distribution between the light exiting surface 120 and the sensor electrode changes. When the human body moves away from the light exiting surface 120, the capacitor ceases to exist between air, the light exiting surface 120, and the sensor electrode. As a result, the total of charges on the sensor electrode decreases, so the electric field distribution between the light exiting surface 120 and the sensor electrode changes once more.

The first sensor 41 and the second sensor 42 of the proximity sensing module 40 pick up changes of the electric field distribution in order to sense whether the skin of the user is touching the present invention. When the light exiting surface 120 is too thick, for example greater than or equal to 3 mm, a distance of the capacitor formed by the human body, the light exiting surface 120, and the sensor electrode is too long. This may create great resistance for the capacitor, and causes the electric field distribution changes to be insignificant, or in other words, causes poor sensitivity for the capacitive proximity sensor toward human touches.

In the present embodiment, the sensor electrode of the first sensor 41 and the sensor electrode of the second sensor 42 are both planar pads. Each of the planar pads has a surface area of 9 mm*12 mm, which equals 108 millimeters squared (mm²). Each of the planar pads also has thickness of only 0.1 mm. The sensor electrodes of the proximity sensing module 40 are made of copper filaments.

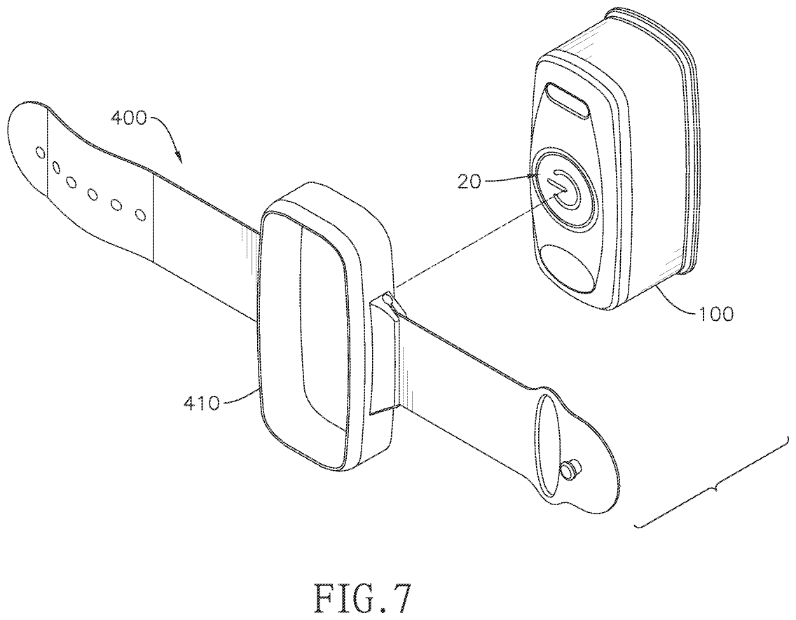

With reference to FIG. 7, a belt strap 400 may be used together with the present invention. The belt strap 400 includes a mount 410, and the mount 410 is used for mounting the shell 100, allowing the shell 100 to be connected to the belt strap 400.

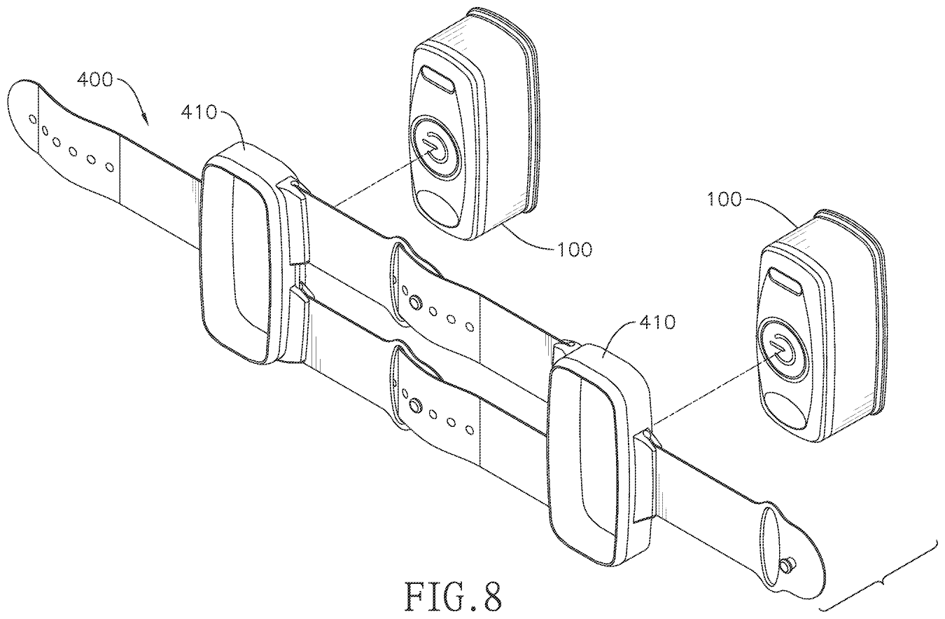

With reference to FIG. 8, in another embodiment, the belt strap 400 includes two mounts 410 for mounting two of the wearable safety lasing light therapy devices of the present invention. The mounts 410 of the belt strap 400 are capable of mounting the shell 100 in multiple quantities, and the belt strap 400 is also strengthened to hold multiple quantities of the shell 100.

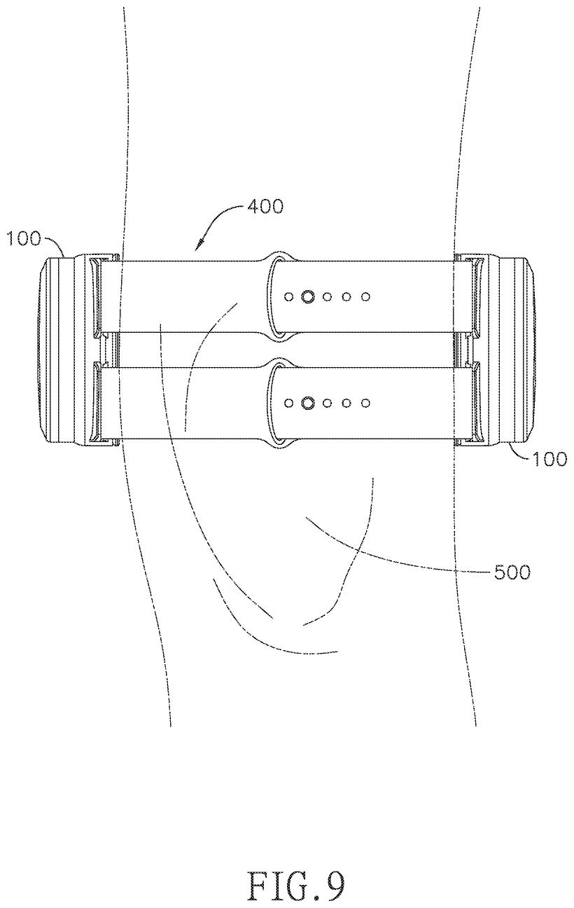

With reference to FIG. 9, when the user uses the belt strap 400 to hold the shell 100 in place, for instance fixing the shell 100 above a knee 500 of the user, the belt strap 400 holds the power switch 20 on the shell 100 facing away from the user. The belt strap 400 also holds the opening 121 and the proximity sensing module 40 facing and closely touching the knee 500. The two mounts 410 are respectively fixed on two sides of the knee 500. Each of the two mounts 410 holds the shell 100, allowing the opening 121 of the shell 100 to aim at acupuncture points of the knee 500 for light therapies. As the first sensor 41 and the second sensor 42 touch the skin of the knee 500, the processing module 10 determines to receive the trigger signal from the proximity sensing module 40, and further allowing the light emission module 30 to generate the light therapy beam to alleviate pain from the knee 500 of the user.

The user of the present invention may freely arrange the belt strap 400 in any position the user sees fit. The belt strap 400 may be fixed on the knee 500, or elsewhere such as a neck, a shoulder, an elbow, a wrist, a waist, or an ankle of the user for conducting the light therapy for bodily alleviations. The said light therapy is a photobiomodulation therapy which uses the light therapy beam to stimulate muscles or tissues around a joint to repair damages and alleviate pain.

The light therapy beam of the present invention is a low energy beam. Light intensity of the low energy beam is still however too strong for human eyes to perceive, and therefore the present invention uses the proximity sensing module 40 as a safety mechanism. When the proximity sensing module 40 of the present invention senses the human body in close proximity of the light exiting surface 120 and sends out the trigger signal, the opening 121 is closely touching the human body, for example the opening 121 is closely touching the knee 500 of the user, and this allows the light therapy beam generated by the light emission module 30 to safely shoot without concerns of accidentally shooting into the eyes of the user. In other words, the processing module 10 needs to first confirm the light emission module 30 is closely touching the knee 500 through the trigger signal of the proximity sensing module 40 before generating the light therapy beam through the light emission module 30. With this safety mechanism, the present invention is able to drastically decrease risks of the light therapy beam shooting directly into the eyes of the user.

What is claimed is:

1. A wearable safety lasing light therapy device, comprising:
a shell, comprising a light exiting surface; wherein the light exiting surface comprises an opening;
a power switch, mounted on the shell;
a light emission module, mounted within the shell and facing the light exiting surface, adapted to produce a light therapy beam; wherein the light therapy beam shoots out of the shell through the opening;
a processing module, mounted within the shell, and electrically connecting the light emission module and the power switch; and
a proximity sensing module, mounted within the shell and facing the light exiting surface, and electrically connecting the processing module;
wherein when the power switch is switched on, the power switch generates a power signal to the processing module;
wherein when the proximity sensing module senses a human body is in close proximity to the light exiting surface, the proximity sensing module generates a trigger signal and sends the trigger signal to the processing module;
wherein when the processing module receives the power signal, the processing module enters a hibernation mode;
wherein when the processing module is in the hibernation mode and receives the trigger signal, the processing module exits the hibernation mode, enters an emission mode, and controls the light emission module to produce the light therapy beam.

2. The wearable safety lasing light therapy device as claimed in claim 1, further comprising:
a control circuit board, mounted within the shell; wherein the processing module is mounted on the control circuit board; wherein the power switch comprises a push button cover and a push button switch; wherein the push button cover is mounted on a front surface of the shell, and the push button switch is mounted on the control circuit board;
a light emission circuit board, mounted within the shell; wherein the light emission module and the proximity sensing module are respectively mounted on the light emission circuit board, and are also respectively facing the light exiting surface of the shell; and
a battery, mounted within the shell and between the control circuit board and the light emission circuit board, and electrically connecting the processing module and the light emission module to provide electricity.

3. The wearable safety lasing light therapy device as claimed in claim 1, further comprising:
a transparent cover, mounted on the light exiting surface of the shell;
wherein the proximity sensing module is an optical sensor; wherein the optical sensor generates a sensing signal and shoots the sensing signal out of the transparent cover for sensing the human body; wherein when the human body approaches the light exiting surface, the optical sensor generates a distance signal and sends the distance signal to the processing module;
wherein the processing module stores a distance threshold; wherein when the processing module determines the distance signal is less than or equal to the distance threshold, the processing module only then determines to receive the trigger signal from the proximity sensing module.

4. The wearable safety lasing light therapy device as claimed in claim 1, wherein the proximity sensing module is a resistive sensor;
wherein when the light exiting surface is deformed and a distance between the light exiting surface and the resistive sensor becomes smaller than a first threshold, the resistive sensor generates the trigger signal and sends the trigger signal to the processing module.

5. The wearable safety lasing light therapy device as claimed in claim 1, wherein the light exiting surface of the shell is an insulator;
wherein the proximity sensing module is a capacitive proximity sensor with a sensor electrode, and the sensor electrode closely contacts the light exiting surface;
wherein when the human body contacts a part of the light exiting surface of the shell corresponding to the sensor electrode, the capacitive proximity sensor generates the trigger signal and sends the trigger signal to the processing module.

6. The wearable safety lasing light therapy device as claimed in claim 2, wherein the proximity sensing module further comprises a first sensor and a second sensor;
wherein the first sensor and the second sensor are respectively mounted on two opposite sides of the light emission circuit board, and are both facing the light exiting surface;
wherein when the first sensor senses the human body is in close proximity to the light exiting surface, the first sensor generates a first trigger signal and sends the first trigger signal to the processing module;
wherein when the second sensor senses the human body is in close proximity to the light exiting surface, the second sensor generates a second trigger signal and sends the second trigger signal to the processing module;
wherein when the processing module is in the hibernation mode and the processing module simultaneously receives the first trigger signal and the second trigger signal, the processing module only then determines to receive the trigger signal from the proximity sensing module.

7. The wearable safety lasing light therapy device as claimed in claim 6, wherein:
after the processing module enters the emission mode, the processing module further determines whether receiving the first trigger signal or the second trigger signal;
when the processing module determines receiving the first trigger signal or the second trigger signal, the processing module stays in the emission mode;
when the processing module determines missing the first trigger signal and the second trigger signal, the processing module exits the emission mode and enters back to the hibernation mode.

8. The wearable safety lasing light therapy device as claimed in claim 2, wherein:
the power switch comprises a backlight light source; the backlight light source is mounted on the control circuit board, and the backlight light source faces the front surface of the shell with the push button cover;
when the processing module enters the hibernation mode, the processing module controls the backlight light source of the power switch to generate a first light signal;
when the processing module enters the emission mode, the processing module controls the backlight light source of the power switch to generate a second light signal;
the first light signal and the second light signal have different colors or different blinking frequencies.

9. The wearable safety lasing light therapy device as claimed in claim 2, wherein:
the light emission module further comprises at least one visible light unit and at least one non-visible light unit;
when the processing module controls the light emission module to generate the light therapy beam, the at least one non-visible light unit generates a laser beam, and the at least one visible light unit generates a colored beam;
the colored beam consists of light in visual spectrum, and the laser beam consists of light outside of visual spectrum.

10. The wearable safety lasing light therapy device as claimed in claim 5, wherein:
a thickness of a wall of the light exiting surface of the shell is less than 3 millimeters (mm).
